(19) United States
(12) Patent Application Publication
(10) Pub. No.: US 2012/0035568 A1
(43) Pub. Date: Feb. 9, 2012

(54) DECORATIVE SANITARY ABSORBENT ARTICLE AND METHOD OF USING SAME

(75) Inventors: Rosa M. Nelson, San Diego, CA (US); Adriana Sandoval, San Diego, CA (US)

(73) Assignees: Adriana Sandoval, San Diego, CA (US); Rosa M. Nelson, San Diego, CA (US)

(21) Appl. No.: 13/206,464

(22) Filed: Aug. 9, 2011

Related U.S. Application Data
(60) Provisional application No. 61/372,048, filed on Aug. 9, 2010.

(51) Int. Cl. A61F 13/47

(52) U.S. Cl. 604/385.01

(57) ABSTRACT

A decorative sanitary absorbent article and a method of using it, includes providing the absorbent article having a decorative appearance or indicia for use in a filmy undergarment having a similar decorative appearance or indicia, to conceal the sanitary absorbent article within the undergarment.

### RELATED APPLICATIONS

This application claims priority to U.S. provisional patent application, Ser. No. 61/372,048, filed Aug. 9, 2010, and entitled “DECORATIVE SANITARY ABSORBENT ARTICLE AND METHOD OF USING SAME.”

### FIELD OF THE INVENTION

The present invention relates in general to a sanitary absorbent article to be worn in an undergarment. It more particularly relates to a sanitary absorbent article with a certain decorative appearance that allows for better concealment of the sanitary absorbent article when it is in use and a method of using the same.

### BACKGROUND ART

There is no admission that the background art disclosed in this section legally constitutes prior art.

Sanitary absorbent articles, such as maxi pads, ultra thin pads, panty liners, adult incontinence pads, and nursing pads are well known and are widely available in the marketplace. Sanitary absorbent articles are designed to absorb and retain fluid from the user's body and to prevent the user's body and clothing from being soiled by this fluid. In use, sanitary absorbent articles are worn in conjunction with an undergarment, interspersed between the user's body and the undergarment.

The majority of sanitary absorbent articles may be referred generally to as sanitary napkins. Sanitary napkins include sanitary absorbent articles, such as maxi pads, ultra thin pads, panty liners, and adult incontinence pads. Sanitary napkins are inserted by the user into the crotch portion of an undergarment, such as underpants.

Typically sanitary napkins are comprised of a main body portion that is elongated with longitudinally opposed front and rear ends and transversally opposed side edges that extend along the length of the sanitary napkin. The main body portion is typically comprised of a permeable top sheet, an impermeable bottom sheet joined to the top sheet, and an absorbent layer encapsulated within the top sheet and the bottom sheet. The impermeable bottom sheet is typically coated with an adhesive for securing the sanitary napkin to the body side of the crotch portion of a user's underpants. In some varieties of sanitary napkins, such as some panty liners, the permeable top sheet may not be present, and the sanitary napkin will be comprised of an absorbent layer overlying an impermeable bottom sheet.

A more recent variety of sanitary napkin, designed to fit the crotch portion of an undergarment with a narrow crotch, such as a thong, is comprised of a main body portion with a general triangular shape, with the front end being larger than the rear end.

Many sanitary napkins are further comprised of at least one pair of lateral foldable attachment panels (wings or flaps) projecting outwardly from the opposed side edges to be folded and fastened to the underside of the crotch portion of the underpants. The impermeable bottom sheet side of the attachment panels are frequently coated with an adhesive for fastening to the non-body side of the crotch portion of a user's underpants.

Another type of sanitary absorbent article is a nursing pad, which is inserted by the user into the cup portions of an undergarment, such as a brassiere.

Nursing pads are typically comprised of a cup-shaped body having a permeable concave top sheet, an impermeable convex bottom sheet joined to the top sheet, and an absorbent layer encapsulated within the top sheet and the bottom sheet. The impermeable convex bottom sheet is typically coated with an adhesive for securing the nursing pad to the body side of the cup portion of a user's brassiere. In some varieties of nursing pads the permeable top sheet may not be present, and the nursing pad will be comprised of an absorbent layer overlying an impermeable bottom sheet.

Often sanitary absorbent articles are not completely concealed within the undergarment when worn, either being visible through the fabric of the undergarment or protruding along the outer edges of the undergarment. Such visibility of the sanitary absorbent article is undesirable to most users of the sanitary absorbent article.

### BRIEF DESCRIPTION OF THE DRAWINGS

The features of this invention and the manner of attaining them will become apparent, and the invention itself will be best understood by reference to the following description of certain embodiments of the invention taken in conjunction with the accompanying drawings, wherein:

FIG. 1 is a pictorial view of a decorative sanitary napkin, according to a preferred embodiment of the present invention, as worn in a typical underpants type of undergarment, showing the certain decorative appearance of the impermeable bottom sheet of the sanitary napkin, which matches the certain decorative appearance of the underpants;

FIG. 2 is a bottom plan view of a preferred embodiment of the decorative sanitary napkin depicted in FIG. 1, showing the certain decorative appearance of the impermeable bottom sheet of the sanitary napkin;

FIG. 3 is a top plan view of a preferred embodiment of the decorative sanitary napkin depicted in FIG. 1, showing the permeable top sheet of the sanitary napkin;

FIG. 4 is a side plan view of a preferred embodiment of the decorative sanitary napkin depicted in FIG. 1, showing a side edge of the sanitary napkin;

FIG. 5 is a pictorial view of a decorative sanitary napkin, according to another preferred embodiment of the present invention, as worn in a typical underpants type of undergarment, showing the certain decorative appearance of the impermeable bottom sheet of the sanitary napkin, which matches the certain decorative appearance of the underpants, and showing the certain decorative appearance of the permeable top sheet of the lateral foldable attachment panels, which matches the certain decorative appearance of the underpants;

FIG. 6 is a bottom plan view of a preferred embodiment of the decorative sanitary napkin depicted in FIG. 5, showing the certain decorative appearance of the impermeable bottom sheet of the sanitary napkin, which does not extend to the lateral foldable attachment panels;

FIG. 7 is a top plan view of a preferred embodiment of the decorative sanitary napkin depicted in FIG. 5, showing the permeable top sheet of the sanitary napkin, with the certain decorative appearance appearing only on the lateral foldable attachment panels;

FIG. 8 is a pictorial view of a decorative sanitary napkin, according to another preferred embodiment of the present invention, as worn in a typical underpants type of undergarment, showing the certain decorative appearance of the impermeable bottom sheet of the sanitary napkin, which matches the certain decorative appearance of the underpants; and

FIG. 9 provides a bottom plan view of a preferred embodiment of the decorative sanitary napkin depicted in FIG. 8, showing the certain decorative appearance of the impermeable bottom sheet of the sanitary napkin.

### DETAILED DESCRIPTION OF CERTAIN EMBODIMENTS OF THE INVENTION

It will be readily understood that the components of the embodiments as generally described and illustrated in the drawings herein, could be arranged and designed in a wide variety of different configurations. Thus, the following more detailed description of the embodiments of the system, components and method of the present invention, as represented in the drawings, is not intended to limit the scope of the invention, as claimed, but is merely representative of the embodiments of the invention.

According to one embodiment of the present invention, there is a decorative sanitary absorbent article for use in a filmy undergarment having a certain decorative appearance or indicia. The decorative sanitary absorbent article comprises an optional permeable top sheet, and an absorbent core and an impermeable bottom sheet with a certain decorative appearance or indicia on the impermeable bottom sheet. The certain decorative appearance on the impermeable bottom sheet is substantially similar to said certain decorative appearance of the undergarment to conceal the sanitary absorbent article within the undergarment.

In one embodiment the sanitary absorbent article is a sanitary napkin, such as a maxi pad, ultra thin pad, panty liner, or adult incontinence pad, wherein the sanitary napkin is inserted by the user into the body side of the crotch portion of the user's underpants. In this embodiment the certain decorative appearance is located on the impermeable bottom sheet of the sanitary napkin.

The nature of the certain decorative appearance on the sanitary napkin may take the form of any certain decorative appearance that may be found on underpants, including, but not limited to one or more of the following: solid colors, lace patterns, polka dot patterns, stripe patterns, floral patterns, leopard skin patterns, zebra skin patterns, geometric shape patterns, text patterns, or any other graphic patterns.

The certain decorative appearance may be applied to the impermeable bottom sheet of the sanitary napkin in a variety of manners, including, but not limited to one or more of the following: direct application of fabric to the impermeable bottom sheet, printing, silk screening, dying, or staining.

In a related embodiment the sanitary napkin may include lateral foldable attachment panels (wings or flaps) that project outwardly from the side edges of the sanitary napkin. In this embodiment the certain decorative appearance on the impermeable bottom sheet of the sanitary napkin may or may not extend to the lateral foldable attachment panels. However, the certain decorative appearance does appear on the lateral foldable attachment panels of the permeable top sheet.

In another embodiment the sanitary absorbent article is a nursing pad, wherein the nursing pad is inserted by the user into the body side of the cup portions of the user's brassiere. In this embodiment the certain decorative appearance is located on the impermeable bottom sheet of the nursing pad.

The nature of the certain decorative appearance on the nursing pad may take the form of any certain decorative appearance that may be found on a brassiere, including, but not limited to one or more of the following: solid colors, lace patterns, polka dot patterns, stripe patterns, floral patterns, leopard skin patterns, zebra skin patterns, geometric shape patterns, text patterns, or any other graphic patterns.

The certain decorative appearance may be applied to the impermeable bottom sheet of the nursing pad in a variety of manners, including, but not limited to one or more of the following: direct application of fabric to the impermeable bottom sheet, printing, silk screening, dying, or staining.

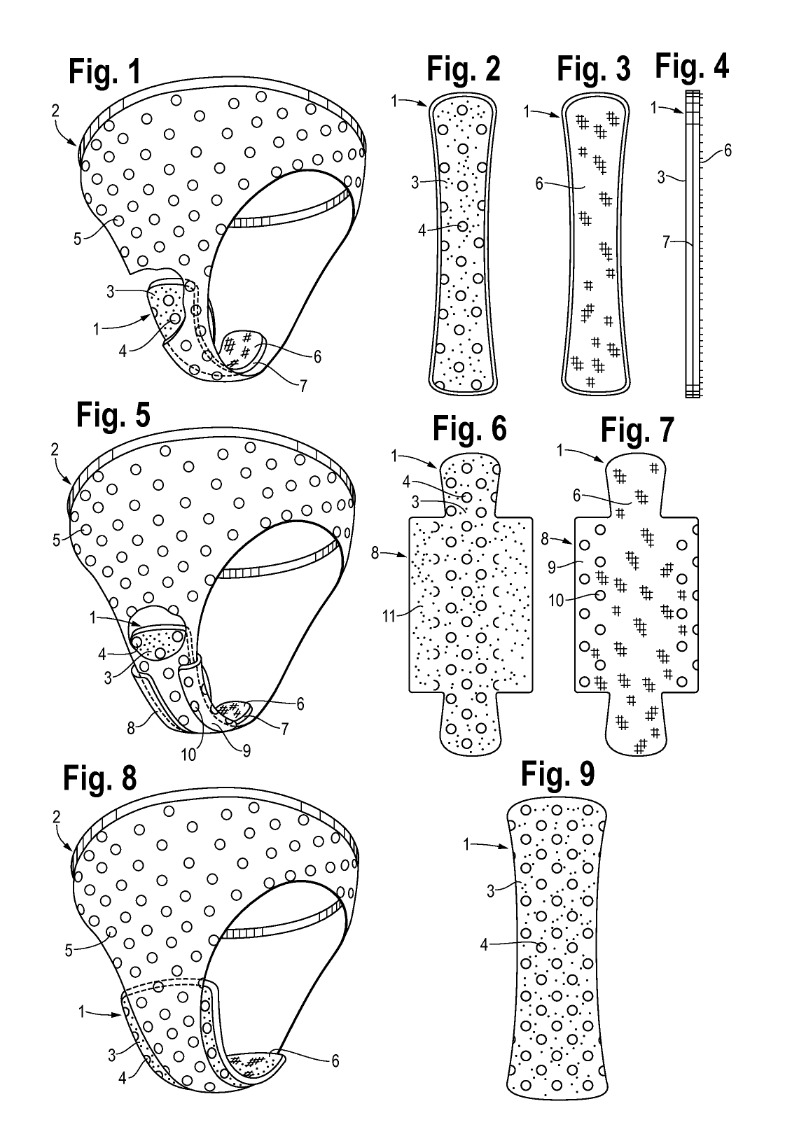

Referring now to FIG. 1, there is depicted a pictorial view of a preferred embodiment of the invention, where a decorative sanitary napkin 1 is shown as used in conjunction with a pair of underpants 2. The impermeable bottom sheet 3 of the decorative sanitary napkin 1 is shown as having a decorative appearance 4 that is substantially the same as the decorative appearance 5 of the non-body side of the underpants 2. The permeable top sheet 6 of the decorative sanitary napkin is shown as not having any decorative appearance. In this embodiment the underpants may be made from a fabric that is not opaque, such as lace, which allows the impermeable bottom sheet of the sanitary napkin to be viewable through the fabric of the underpants.

FIG. 2 is a bottom plan view of a preferred embodiment of the decorative sanitary napkin 1 of FIG. 1, showing the certain decorative appearance 4 of the impermeable bottom sheet 3 of the sanitary napkin 1.

FIG. 3 is a top plan view of a preferred embodiment of the decorative sanitary napkin 1 of FIGS. 1 and 2, showing the permeable top sheet 6 of the sanitary napkin 1, wherein the permeable top sheet 6 does not have a decorative appearance.

FIG. 4 is a side plan view of a preferred embodiment of the decorative sanitary napkin 1 of FIGS. 1, 2 and 3, showing a side edge 7 of the sanitary napkin.

In FIG. 5, there is depicted a pictorial view of another preferred embodiment of the invention, where a decorative sanitary napkin 1 is shown as used in conjunction with a pair of underpants 2. The impermeable bottom sheet 3 of the decorative sanitary napkin 1 is shown as having a decorative appearance 4 that is substantially the same as the decorative appearance 5 of the non-body side of the underpants 2. The permeable top sheet 6 of the decorative sanitary napkin is shown as not having any decorative appearance. The sanitary napkin of this embodiment further includes a pair of lateral foldable attachment panels 8, wherein the permeable top sheet 9 of the lateral foldable attachment panels 8 is shown as having a decorative appearance 10 that is substantially the same as the decorative appearance 5 of the non-body side of the underpants 2.

FIG. 6 is a bottom plan view of a preferred embodiment of the decorative sanitary napkin 1 of FIG. 5, showing the certain decorative appearance 4 of the impermeable bottom sheet 3 of the sanitary napkin 1. The sanitary napkin of this embodiment further includes a pair of lateral foldable attachment panels 8, wherein the impermeable bottom sheet 11 of the lateral foldable attachment panels 8 is shown as not having any decorative appearance. However, in some embodiments, not shown here, there may be a decorative appearance on the impermeable bottom sheet of the lateral foldable attachment panels.

FIG. 7 is a top plan view of a preferred embodiment of the decorative sanitary napkin 1 of FIGS. 5 and 6, showing the permeable top sheet 6 of the sanitary napkin 1, wherein the permeable top sheet 6 does not have a decorative appearance. The sanitary napkin of this embodiment further includes a pair of lateral foldable attachment panels 8, wherein the permeable top sheet 9 of the lateral foldable attachment panels 8 is shown as having a decorative appearance 10.

Referring now to FIG. 8, there is depicted a pictorial view of a preferred embodiment of the invention, where a decorative sanitary napkin 1 is shown as used in conjunction with a pair of underpants 2. The impermeable bottom sheet 3 of the decorative sanitary napkin 1 is shown as having a decorative appearance 4 that is substantially the same as the decorative appearance 5 of the non-body side of the underpants 2. The permeable top sheet 6 of the decorative sanitary napkin is shown as not having any decorative appearance. In this embodiment the underpants may be made from a fabric that is opaque, which may substantially hide the sanitary napkin from view except for where the sanitary napkin protrudes from the edge of the underpants.

FIG. 9 is a bottom plan view of a preferred embodiment of the decorative sanitary napkin 1 of FIG. 8, showing the certain decorative appearance 4 of the impermeable bottom sheet 3 of the sanitary napkin 1.

In accordance with another disclosed embodiment of the invention, there is provided a method of concealing from view a sanitary absorbent article within an undergarment having a certain decorative appearance. The method includes: providing a decorative sanitary absorbent article having a distinctive appearance on the undergarment facing surface of an impermeable bottom sheet and an absorbent layer overlying the non-undergarment facing surface of the impermeable bottom sheet and an optional permeable top sheet overlying the absorbent layer, where the distinctive appearance on the impermeable bottom sheet is substantially similar to the certain decorative appearance of the undergarment to conceal the sanitary absorbent article from view when worn by the user; and instructing the user to insert the decorative sanitary absorbent article within the undergarment with the undergarment facing surface of the impermeable bottom sheet overlying the body side of the undergarment to conceal the sanitary absorbent article from view when worn by the user.

While particular embodiments of the present invention have been disclosed, it is to be understood that various different modifications are possible and are contemplated within the true spirit and scope of the appended claims. There is no intention, therefore, of limitations to the exact abstract or disclosure herein presented.

What is claimed is:

1. A decorative sanitary absorbent article for use in a filmy undergarment with a certain decorative appearance, comprising:
an absorbent layer and an impermeable bottom sheet with a certain decorative appearance on the impermeable bottom sheet,
wherein the certain decorative appearance on the impermeable bottom sheet is substantially similar to the certain decorative appearance of the undergarment to conceal the sanitary absorbent article from view within the undergarment.

2. An article according to claim 1, further including a top sheet.

3. An article according to claim 1, wherein the decorative appearance being applied to the impermeable bottom sheet by at least one of direct application, printing, silk screening, dying, and staining.

4. An article according to claim 1, further including projecting outwardly lateral foldable attachment panels.

5. An article according to claim 2, wherein the impermeable bottom sheet of the attachment panels does not bear the decorative appearance.

6. An article according to claim 5, wherein the permeable top sheet of the attachment panels bears the decorative appearance.

7. An article according to claim 1, wherein the bottom sheet is sized to protrude on either side from the undergarment.

8. A method of concealing from view a sanitary absorbent article within a filmy undergarment having a certain decorative appearance, comprising:
providing a decorative sanitary absorbent article having a distinctive appearance on the undergarment facing bottom surface of an impermeable bottom sheet and an absorbent layer overlying the non-undergarment facing surface of the impermeable bottom sheet and an optional permeable top sheet overlying the absorbent layer;
wherein the distinctive appearance on the bottom surface of the impermeable bottom sheet is substantially similar to the certain decorative appearance of the undergarment to conceal the sanitary absorbent article from view when worn by the user; and
instructing the user to insert the decorative sanitary absorbent article within the undergarment with the undergarment facing surface of the impermeable bottom sheet overlying the body side of the undergarment to conceal the sanitary absorbent article from view when worn by the user.

9. A method of making a sanitary absorbent article for use within a filmy undergarment having a certain decorative indicia thereon, comprising:
securing an absorbent layer to a top surface of an impermeable bottom sheet, wherein the bottom sheet has decorative indicia on the bottom surface thereof to match the certain decorative indicia on the outer surface of the undergarment to conceal from view the sanitary article when worn by the user.
